(19) United States
(12) Patent Application Publication
(10) Pub. No.: US 2014/0277058 A1
(43) Pub. Date: Sep. 18, 2014

(54) AIRWAY DILATION SHAFT WITH STAGGERED ADJACENT INTERNAL LUMENS

(71) Applicant: ACCLARENT, INC., Menlo Park, CA (US)

(72) Inventor: Show-Mean Wu, Fremont, CA (US)

(21) Appl. No.: 13/795,791

(22) Filed: Mar. 12, 2013

(51) Int. Cl. A61M 29/02; A61M 25/09

(52) U.S. Cl. CPC A61M 29/02; A61M 25/09
USPC 606/192

(57) ABSTRACT

A plurality of tubes is combined to form a tube assembly, which may be used as an airway dilation shaft. The tubes define lumens that extend along parallel axes that are laterally offset from each other. The tubes may be contained within an outer tube or within a shrink wrap. Different functions may be achieved by the different lumens, such as inflating an airway dilation balloon, carrying oxygen or suction, delivering drugs or other substances, and carrying a guide wire. The hardness, inner diameter, outer diameter, and/or wall thickness of each tube may vary along its length. The tubes may distally terminate at different longitudinal positions. A first tube may be disposed within a second tube, with a third tube being external to yet parallel with the first and second tubes. The tubes may be tested for integrity before or after they are combined to form a tube assembly.

### BACKGROUND

In some instances, it may be desirable to dilate an anatomical passageway in a patient. This may include dilation of ostia of paranasal sinuses, dilation of a patient's airway (e.g., to treat a stenosis within the larynx), dilation of the nasal cavity, dilation of the Eustachian tube, dilation of other passageways within the ear, nose, or throat, dilation of blood vessels, dilation of the urethra, etc. One method of dilating anatomical passageways includes using a guide wire and catheter to position an inflatable balloon within the anatomical passageway, then inflating the balloon with a fluid (e.g., saline) to dilate the anatomical passageway.

Airway stenosis (or “airway narrowing”) is a medical condition that occurs when some portion of a patient's airway becomes narrowed or constricted, thus making breathing difficult. A stenosis may occur in any part of the airway including the larynx, trachea, bronchi, or a combination of any of the above mentioned regions. Both adults and children may develop a stenosis. In some instances, a stenosis is caused by intubation, which is when a tube is placed in the airway for ventilation/breathing assistance in a patent who cannot breathe. Intubation for prolonged periods of time may traumatize the airway, causing scar tissue formation that forms the stenosis.

Therapies for treating an airway stenosis range from endoscopic treatments, such as dilation and laser resection, to open procedures, such as laryngotracheal reconstruction. In one technique, a series of rigid dilators of increasing diameter are pushed down the airway, gradually expanding the constriction but also applying shear forces to the airway. Balloon catheters may also be used to perform dilation of an airway or other anatomical passageway. For instance, the expandable balloon may be positioned within a stenosis in an airway (e.g., larynx, trachea, bronchi, etc.) and then be inflated, to thereby dilate the airway and increase airflow. The dilated airway may then allow for improved breathing. An example of a system that may be used to perform such procedures is described in U.S. Pub. No. 2010/0168511, entitled “System and Method for Dilating an Airway Stenosis,” published Jul. 1, 2010, the disclosure of which is incorporated by reference herein.

While several airway dilation systems have been made and used, it is believed that no one prior to the inventor(s) has made or used the invention described in the appended claims.

### BRIEF DESCRIPTION OF THE DRAWINGS

While the specification concludes with claims that particularly point out and distinctly claim this technology, it is believed this technology will be better understood from the following description of certain examples taken in conjunction with the accompanying drawings, in which like reference numerals identify the same elements and in which:

FIG. 1 depicts a side view of an exemplary system for dilating a stenosis in the airway, including a balloon catheter and a stylet;

FIG. 2 depicts a side view of the stylet of FIG. 1;

FIG. 3A depicts a cross sectional view of the system of FIG. 1 being introduced into an airway, with the balloon positioned at a stenosis in a collapsed state;

FIG. 3B depicts a cross sectional view of the system of FIG. 3A, with the balloon inflated to a dilated state;

FIG. 4 depicts a cross-sectional end view of an exemplary three-lumen catheter assembly for use in the system of FIG. 1;

FIG. 5 depicts a cross-sectional end view of another exemplary three-lumen catheter assembly for use in the system of FIG. 1;

FIG. 6 depicts a cross-sectional end view of yet another exemplary three-lumen catheter assembly for use in the system of FIG. 1;

FIG. 7 depicts a side elevational view of still another exemplary three-lumen catheter assembly for use in the system of FIG. 1;

FIG. 8 depicts a side elevational view of an exemplary tube for use in a catheter assembly;

FIG. 9 depicts a cross-sectional view of the tube of FIG. 8, taken along line 9-9 of FIG. 8;

FIG. 10 depicts a cross-sectional view of the tube of FIG. 8, taken along line 10-10 of FIG. 8;

FIG. 11 depicts a side elevational view another exemplary tube for use in a catheter assembly;

FIG. 12 depicts a cross-sectional view of the tube of FIG. 10, taken along line 12-12 of FIG. 11;

FIG. 13 depicts a cross-sectional view of the tube of FIG. 11, taken along line 13-13 of FIG. 11;

FIG. 14 depicts a cross-sectional end view of another exemplary three-lumen catheter assembly for use in the system of FIG. 1;

FIG. 15 depicts a flowchart illustrating an exemplary process of testing, combining, and using single lumens in combination; and

FIG. 16 depicts a flowchart illustrating an exemplary process of combining, individually testing, and using single lumens in combination.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

### I. OVERVIEW OF EXEMPLARY BALLOON DILATION CATHETER SYSTEM

FIG. 1 shows an exemplary dilation catheter system (8), which may be used to dilate a stenosis in an airway; or to dilate some other anatomical passageway (e.g., within the ear, nose, throat, cardiovascular system, etc.). At least part of system (8) may be constructed and operable in accordance with at least some of the teachings of U.S. Pub. No. 2010/0168511, the disclosure of which is incorporated by reference herein. It should be understood that dilation catheter system (8) may be used to dilate either a naturally occurring passageway in a patient or a surgically created passageway in a patient.

Dilation catheter system (8) of this example comprises a balloon catheter (10) and a stylet (22). Balloon catheter (10) comprises a shaft (12) positioned between a hub (14) and a balloon (18). Balloon (18) is coupled to a distal end of shaft (12) and is configured to receive fluid through balloon catheter (10). Stylet (22) is slidably positioned through balloon catheter (10). In some versions, at least a portion of stylet (22) has a greater stiffness than at least a portion of balloon catheter (10), such that when stylet (22) is bent and inserted within balloon catheter (10), balloon catheter (10) at least partially conforms to the shape of stylet (22). In a dilation procedure, stylet (22) is used to advance balloon catheter (10) within an airway or targeted anatomical passageway (e.g., at a stenosis site). Balloon (18) may then be actuated to an expanded state to open or dilate the targeted anatomical passageway. Balloon (18) may then be actuated back to a collapsed state such that balloon (18) is deflated. This process may be repeated to dilate several anatomical passageways.

A. Exemplary Balloon Catheter

As shown in FIG. 1, balloon catheter (10) comprises a catheter shaft (12). An inflatable balloon (18) is attached to a distal end of shaft (12) via adhesive or other attachment means. A hub (14) is coupled to a proximal end of shaft (12) and comprises a stylet port (38) and an inflation port (37). Stylet (22) is inserted within stylet port (38) and generally resides within an inner lumen of shaft (12). Fluid (e.g., saline, etc.) is introduced through inflation port (37) through shaft (12) to inflate balloon (18).

Balloon catheter (10) may have any number of suitable sizes, shapes and configurations. For example, balloon (18) may have different lengths and diameters in different embodiments, to accommodate different patient anatomies. The overall catheter (10) length and diameter may also vary. For example, the overall length of balloon catheter (10) (i.e., from the proximal end of hub (14) to the distal end of catheter shaft (12)) is about 35-70 cm, such as less than or equal to about 50 cm, or about 45 cm.+−0.5 cm. Catheter (10) may be handled and manipulated with one hand. The working length of balloon (18) in FIG. 1 is about 40 mm+/−0.2 mm. By “working length” it is meant the length between the two tapered portions of balloon (18). In some versions, the working length of balloon (18) may range from between about 10 mm and about 60 mm such as about 16-45 mm. The outer diameter of the fully inflated working length of balloon (18) may also vary. In the present example, balloon (18) has an inflated diameter of about 14.1 mm+/−0.5 mm. In some versions, balloon (18) diameter may range from about 3 mm to about 24 mm, such as about 5-15 mm. A combination of balloon diameters and lengths may be provided, such that a physician may choose an appropriate size for an adult or pediatric patient. In one example, the following balloon diameters and lengths may be provided: 5 mm by 24 mm; 7 mm by 24 mm; 10 mm by 40 mm; and 14 mm by 40 mm. Of course, any of a number of other combinations of sizes of balloons (58) may be provided.

Any suitable material may be used to form balloon (18). Balloon (18) may be compliant, semi-compliant or non-compliant. Balloon (18) may be made of nylon, some other polymer, such as PTFE, and/or any other suitable material(s). In some versions, balloon (18) is formed of an elastic/extensible material that is resiliently biased to assume a shrunken, non-inflated configuration, such that the material forming balloon (18) is under increased tension when balloon (18) is in a non-deflated state. In some other versions, balloon (18) is formed of a material that is flexible yet substantially inelastic/non-extensible, such that the material forming balloon does not provide a significant resilient bias. In other words, balloon (18) does not stretch in response to increased fluid pressure inside balloon (18), even though the effective outer diameter of balloon (18) increases in response to increased fluid pressure. Such inelastic versions of balloon (18) may nevertheless be filled with fluid, with the fluid pressure being increased to provide an outwardly directed force via balloon (18), and this process may be referred to as “inflating.” When the pressure of fluid inside balloon (18) is reduced, this process may be referred to as “deflating,” even if the material forming balloon (18) does not elastically shrink, since balloon (18) may nevertheless flexibly collapse in response to reduced fluid pressure. Thus, it should be understood that the use of terms like “inflate,” “inflated,” “deflate,” and “deflated” does not necessarily mean that the material forming balloon (18) undergoes any elastic stretching or shrinking as the fluid pressure within balloon (18) changes.

In some versions, balloon (18) may include an outer slip-resistant surface, which may be formed by a textured surface or a coating. Such a surface may help prevent slipping of balloon (18) out of an airway structure during inflation and/or may facilitate re-wrapping balloon (18) by hand after deflation if balloon (18) is to be used for a second or subsequent dilation procedure. Examples of such balloons are provided in U.S. patent application No. [FBT DOCKET NO. ACC5059USPSP.600452], entitled “Features to Enhance Grip of Balloon within Airwary,” filed on a date even herewith, the disclosure of which is incorporated by reference herein.

Catheter shaft (12) may also be formed of any suitable material. It may be desirable to form shaft (12) from material(s) selected so that shaft (12) is unlikely to kink when bent, such as when bent by stylet (22) and/or a user. One such material, for example, is Pebax, although other polymers may be used. Shaft (12) may also have any suitable color and may include one or more shaft markings. The shaft color and markings may be built into shaft (12) by using a colored material or may be added by applying paint or another colorant. In some versions, shaft (12) may have a dark color, such as black or dark blue, and one or more light colored markings may be applied over the dark shaft (12). In some versions, the markings (not shown) may include direct visualization markings (viewed directly with the naked eye or an endoscope) and/or radiographic markings (viewed with a radiographic device such as intraoperative fluoroscopy). Any suitable combination, size and color of markings may be used. One example of shaft color and shaft markings, which could be used or modified for balloon catheter (50), is the Relieva Solo Pro™ Sinus Balloon Catheter, manufactured by Acclarent, Inc. of Menlo Park, Calif.

B. Exemplary Stylet

FIG. 2 shows stylet (22) in greater detail. Stylet (22) comprises a core member (26) with a proximal section (28) and a distal section (30). A coil (32) is disposed around at least part of distal section (30) of core member (26). A luer lock member (35) is coupled with a proximal end of core member (26) for coupling with a hub on balloon catheter (10). In some versions, stylet (22) does not include a coil (32). Core member (26) and/or coil (32) may be formed of nitinol, stainless steel, or other biocompatible materials. Distal portion (30) of stylet (22) includes a bend or curve (34) that is stiff enough to bend balloon catheter (10) during the placement of balloon catheter (10) within the airway of the patient. In some versions, stylet (22) may be provided in a generally straight configuration. Stylet (22) may be pre-formed to have a bend (34), or stylet (22) may be malleable, such that a user may bend stylet (22) and stylet (22) maintains the user-created bend. This malleability allows a user to adjust a bend angle according to the airway anatomy of a particular patient. Proximal section (28) of stylet (22) may be generally stiff, a distal section (30) may be generally malleable, and an extreme distal portion may be atraumatic and very flexible or even floppy. This variation in flexibility along the length of stylet (22) may be achieved by using different materials, such as stainless steel and nitinol. Alternatively, one material, such as stainless steel, may be used and the diameter of stylet (22) may be altered to achieve the variation in flexibility along the length of stylet (22).

Stylet (22) has an overall length approximately as long or slightly longer than balloon catheter (10). In some versions, stylet (22) includes an atraumatic, flexible distal tip portion that extends distally out of balloon catheter (10) when stylet (22) is fully disposed within catheter (10). This tip portion may be, for example, between about 0.25 cm to about 8 cm (e.g., about 1-5 cm) in length; and may facilitate the ability of a user to advance system (8) through a patient's airway atraumatically. The overall length of stylet (22) may vary from about 30 cm to about 80 cm, such as from about 45 cm to about 60 cm. Of the overall length, a flexible distal portion of stylet (22) may be from about 5-20 cm, such as from about 10-15 cm. Bend (34) may have any suitable angle, such as from greater than 0 degrees to about 20 degrees. The diameter of stylet (22) may be less than about 1.3 mm, such as 0.9 mm or less. The diameter may decrease distally to about 0.13 mm+/−0.013 mm. Of course, the foregoing dimensions are mere examples. Any other suitable dimensions may be used.

Stylet (22) may be attached to balloon catheter (10), or stylet (22) may be removably connected to balloon catheter (10). Stylet (22) comprises a luer lock member (35) with threads on proximal section (28) that screw into opposing threads disposed on a luer (36) of balloon catheter (10). In some versions, balloon catheter (10) may include a locking mechanism (not shown) to lock stylet (22) in position within catheter (10). The locking mechanism can be any mechanical device, including a lever, a ball and pin, a luer, etc. All or part of distal section (30) of stylet (22) may extend out of the distal end of catheter (10). Stylet (22) may be locked to balloon catheter (10) at different positions or lengths so the distal end of stylet (22) extends out of or is positioned within balloon catheter (10) at different lengths. The length, diameter(s) and stiffness characteristics of stylet (22) may be varied in different embodiments to confer different performance characteristics to the overall system (8).

Use of stylet (22) to insert balloon catheter (10) helps to guide the distal end of balloon catheter (10) through the airway of the patient and to the stenotic region. Stylet (22) provides increased steerability during advancement of balloon catheter (10). Torquability of balloon catheter (10) is also increased when using stylet (22). In some versions, luer lock member (35) of stylet (22) and luer (36) of balloon catheter (10) mate together, so that stylet (22) and balloon catheter (10) may be rotated together and thus steered into a constricted portion of an airway.

In some versions, stylet (22) may have a light emitting portion, such as a light emitting distal end or tip. For example, stylet (22) may include one or more light fibers to transmit light from a light source attached to the proximal end of stylet (22) to its distal end. Light from a light emitting stylet (22) may be used to help a user visualize a patient's airway from the inside using a scope and/or in some cases from the outside via transillumination through the patient's skin. A light emitting guidewire device that may be used or modified to achieve such an illuminating stylet (22) is the Relieva Luma™ Sinus Illumination Guidewire/System, manufactured by Acclarent, Inc. of Menlo Park, Calif. Such an illuminating stylet (22) may have any of the features described above with the additional feature of light emitting capability.

C. Exemplary Method of Use of the System

FIGS. 3A and 3B show a method for dilating an stenotic region (4) in an airway (2), such as in a case of subglottic stenosis. Dilation system (8) is introduced through the mouth and into the airway of the patient. Optionally, a bronchoscope (not shown) or other scope device may be used to visualize the positioning of dilation system (8). Dilation system (8) may be bent either by the user or by the manufacturer of system (8). For example, stylet (22) may be bent and then inserted into balloon catheter (10), while in other cases stylet (22) and balloon catheter (10) may be bent together, with stylet (22) already residing in catheter (10). The support of stylet (22) and the bend in the overall system (8) may help a physician navigate system (8) through the patient's airway to position balloon (18) within at least a portion of stenotic region (4). As shown in FIG. 3A, inflatable balloon (18) of the catheter (10) is in an unexpanded configuration during advancement and placement of balloon catheter (10). As shown in FIG. 3B, once balloon (18) is positioned within stenotic region (4) of the airway (2), inflatable balloon (18) is inflated to dilate stenotic region (4). Balloon (18) is then deflated to enable removal from airway (2). By way of example only, balloon (18) may be deflated by actively drawing the fluid from balloon (18); by venting the fluid in balloon (18), allowing the inward pressure imposed by airway (2) to drive fluid from balloon (18); or in any other suitable fashion as will be apparent to those of ordinary skill in the art in view of the teachings herein.

In some versions, stylet (22) remains in balloon catheter (10) during inflation of balloon (18). Maintaining stylet (22) in catheter (10) during inflation may give catheter (10) added column strength and help maintain the position of balloon (18) within stenotic region (4), thus avoiding slipping. In some versions, stylet (22) is removed from balloon catheter (10) before inflating. Stylet (22) may be removed from balloon catheter (10) after balloon catheter (10) is properly positioned within airway (2) of the patient, or stylet (22) can be removed after stenosis (4) has been dilated but before removing balloon catheter (10) from the patient.

Inflatable balloon (18) may be inflated more than once to dilate stenotic region (4) of airway (2). The physician inflates inflatable balloon (18) to a desired pressure during each dilation of stenosis (4). Proper dilation of stenotic region (4) can be confirmed by visualizing the region with the bronchoscope/endoscope.

### II. EXEMPLARY ALTERNATIVE TUBE ASSEMBLY

The following examples relate to tube assemblies that serve as variations of balloon catheter (10). It should be understood that the following examples may provide enhanced effects over a conventional balloon catheter (10). By way of example only, the following examples may provide enhanced resistance to kinking, enhanced collapse strength, and/or other effects. In some versions, these enhanced effects are provided by separating the axes of lumens within a tube assembly. In other words, separating lumen axes and orienting them in an offset manner may provide enhanced effects not provided by conventional balloon catheters (10). Various other effects that may be provided by separating lumen axes will be apparent to those of ordinary skill in the art in view of the teachings herein. Furthermore, it should be understood that the following examples are merely illustrative, and that other variations will also be apparent to those of ordinary skill in the art in view of the teachings herein.

FIG. 4 shows an exemplary alternative tube assembly (100) that may be used as a variation of balloon catheter (10). In particular, tube assembly (100) comprises a plurality of individual tubes (110, 120, 130) having respective lumens (112, 122, 132) extending along respective axes that run in parallel with each other. While three tubes (110, 120, 130) are provided in the present example, it should be understood that any other suitable number of tubes (110, 120, 130) may be used. For instance, more than three or less than three tubes (110, 120, 130) may be used. In some versions, each tube (110, 120, 130) has a distinct purpose, while in others, multiple tubes (110, 120, 130) share the same function. In the present example, lumen (112) of tube (110) provides a dedicated path for communicating saline or some other fluid to inflatable balloon (18) at the distal end of tube assembly (100). Lumen (122) of tube (120) provides a dedicated path for communicating a drug to an opening within the airway of the patient. Such a drug delivery opening may be positioned at or near inflatable balloon (18) or elsewhere. Lumen (132) of tube (130) provides a dedicated path for insertion of stylet (22), a guide wire, or some other type of structure to guide and/or otherwise manipulate tube assembly (100) in the patient's airway. Tubes (110, 120, 130) may be secured together using adhesives, heat welding, ultrasonic welding, and/or using any other suitable techniques.

FIG. 5 shows an exemplary alternative tube assembly (200) formed by three inner tubes (210, 220, 230) and an outer tube (240). Tubes (210, 220, 230) all extend through lumen (242) of outer tube (240) and are arranged about the longitudinal axis of outer tube (240). Tubes (210, 220, 230) have respective lumens (212, 222, 232) extending along respective axes that run in parallel with each other. While three inner tubes (210, 220, 230) are provided in the present example, it should be understood that any other suitable number of tubes (210, 220, 230) may be used. It should also be understood that inner tubes (210, 220, 230) may have shared and/or distinct purposes, similar to tubes (110, 120, 130) discussed above. Also like tubes (110, 120, 130), tubes (210, 220, 230) may be secured together in any suitable fashion. Alternatively, one or more of tubes (210, 220, 230) may be detached relative to the other tubes (210, 220, 230) along at least a portion of the length of tubes (210, 220, 230). It should also be understood that tubes (210, 220, 230) may be secured to outer tube (240), if desired, using any suitable techniques. In some versions, the remaining gap in lumen (242) is used to carry negative pressure to provide suction at a work site. In addition or in the alternative, the remaining gap in lumen (242) may be used to deliver a drug or other substance to the work site. Other suitable uses will be apparent to those of ordinary skill in the art in view of the teachings herein.

FIG. 6 shows yet another exemplary alternative tube assembly (300) formed by three inner tubes (310, 320, 330) and an outer wrap (340). Tubes (310, 320, 330) have respective lumens (312, 322, 332) extending along respective axes that run in parallel with each other. While three inner tubes (310, 320, 330) are provided in the present example, it should be understood that any other suitable number of tubes (310, 320, 330) may be used. It should also be understood that inner tubes (310, 320, 330) may have shared and/or distinct purposes, similar to tubes (310, 320, 330) discussed above. Outer wrap (340) may comprise a conventional wrap that shrinks in response to heat. Thus, after tubes (310, 320, 330) are arranged as shown, outer wrap (340) may be positioned over tubes (310, 320, 330) and then be heated to shrink about tubes (310, 320, 330), thereby providing a form fit around tubes (310, 320, 330). In some such instances, this form fitting of outer wrap (340) about tubes (310, 320, 330) substantially holds tubes (310, 320, 330) together. Thus, in some such versions, an adhesive, welding, or other technique is not used to further secure tubes (310, 320, 330) together. It should be understood that outer wrap (340) may extend along the full length or just a portion of the length of tubes (310, 320, 330).

FIG. 7 shows an exemplary tube assembly (400) that is also formed by three tubes (410, 420, 430). Tubes (410, 420, 430) may be formed similar to any other tubes described herein. Tubes (410, 420, 430) of this example have respective distal ends (414, 424, 434) that terminate at different longitudinal positions along the length of tube assembly (400). This may provide an effective outer diameter of tube assembly (400) that varies along the length of tube assembly (400)—being larger at the proximal end of tube assembly (400) and smaller at the distal end of tube assembly (400). Such a varying effective outer diameter may provide some degree of gradual dilation of the anatomical passageway as tube assembly (400) is driven through the anatomical passageway. In addition or in the alternative, staggering the distal ends (414, 424, 434) may provide a desired staggering of exit points for tubes (410, 420, 430) within the anatomical passageway. For instance, if tube (430) is used to inflate a balloon (18), tube (414) may be used to deliver a drug, an instrument, or something else proximal to balloon (18). Varying the termination positions or lengths of tubes (410, 420, 430) may also facilitate manipulation of a stylet (22), guide wire, scope, oxygen source, and/or other devices. Other suitable uses and functionalities for staggered distal ends (414, 424, 434) will be apparent to those of ordinary skill in the art in view of the teachings herein.

It should also be understood that the tubes of a tube assembly may be tapered along their length. For instance, FIGS. 8-10 show an exemplary tube (510) that may be readily incorporated into any of the tube assemblies described herein. The outer diameter of the proximal end (516) of tube (510) is greater than the outer diameter of the distal end (514) of tube (510), such that the outer diameter of tube (510) tapers downwardly along the length of tube (510). As best seen in FIGS. 9-10, the lumen (512) defined by tube (510) also has a diameter that is greater at the proximal end (516) of tube than the diameter of lumen (512) at the distal end (514) of tube. It should therefore be understood that the sidewall of tube (510) has a uniform thickness along the length of tube (510).

FIGS. 11-13 show another exemplary tapered tube (610) that may be readily incorporated into any of the tube assemblies described herein. Like tube (510), the outer diameter of the proximal end (616) of tube (610) is greater than the outer diameter of the distal end (614) of tube (610), such that the outer diameter of tube (610) tapers downwardly along the length of tube (610). Unlike tube (510), however, the sidewall thickness of tube (610) is not uniform along the length of tube (610). Instead, the thickness of the sidewall of tube (610) decreases along the length of tube (610) in order to provide a constant diameter for lumen (612) along the length of tube (610). Still other suitable ways of providing a tapered tube in a tube assembly will be apparent to those of ordinary skill in the art in view of the teachings herein. It should also be understood that incorporating one or more tapered tubes into a tube assembly may provide a taper for the tube assembly itself. As noted above with respect to the effective taper provided by tube assembly (400), a tapered tube assembly may provide some degree of gradual dilation of the anatomical passageway as the tapered tube assembly is driven through the anatomical passageway. Such a taper may also enhance structural integrity of the tube system, thereby reducing risks of kinking, buckling, collapsing, etc., during use of the tube system.

FIG. 14 shows another exemplary tube assembly (700) formed by two outer tubes (710, 730) and an inner tube (720). Tubes (710, 720, 730) have respective lumens (712, 722, 732). Tube (720) is coaxially disposed within tube (710) in this example, such that lumens (712, 720) are coaxial. Tube (730) is laterally offset from tube (710), such that lumen (732) extends along an axis that is parallel to the axis shared by lumens (712, 722). While three tubes (710, 720, 730) are provided in the present example, it should be understood that any other suitable number of tubes (710, 720, 730) may be used in any other suitable arrangement. It should also be understood that inner tubes (710, 720, 730) may have shared and/or distinct purposes, similar to tubes (710, 720, 730) discussed above. In some versions, tube (720) is positioned within tube (710) but is not coaxial with tube (710). For instance, tube (720) may be secured to the interior surface of the sidewall of tube (710), with lumens (712, 722) still extending along parallel axes. It should also be understood that tubes (710, 730) may be secured together using an adhesive/welding/etc.; that a wrap similar to wrap (340) may be provided about tubes (710, 730); and that am outer tube similar to outer tube (240) may be provided about tubes (710, 730).

In any of the tube assemblies described herein, the hardness of the sidewall of one or more tubes in the tube assembly may vary along its working length. For instance, a distal section of one or more tubes of a tube assembly may be more flexible than a proximal section of the same tubes. In some versions, one or more tubes of a tube assembly is/are formed by an axially aligned stack of two or more lumen segments. By way of example only, at least one of the tubes of a tube assembly may be formed by an axially aligned stack of two or more lumen segments that are joined together by couplings. One merely illustrative example of a coupling is a “bump tube” coupling, which comprises a nipple-like tube that is insertable into the lumen of a tube and has an annular flange that provides friction and a seal against the inner diameter of the lumen. Alternatively any other suitable type of coupling may be used.

Also in any of the tube assemblies described herein, one or more tubes forming the tube assembly may include indicia that provide an operator with visual feedback indicating the depth of insertion of the tube assembly within the patient. For instance, one or more tubes forming the tube assembly may include a series of graduations that are exposed relative to the patient based on the depth to which the tube assembly is inserted in the patient. Such graduations may include numbers indicating a value associated with the depth of insertion. As yet another merely illustrative example, the indicia may include color-coded features, with different colors being associated with different depths of insertion. Other suitable forms that indicia may take will be apparent to those of ordinary skill in the art in view of the teachings herein.

Various examples disclosed herein may enable easier construction and/or testing of multi-lumen tube assemblies. For instance, individual tubes may be easily fabricated with tightly controlled dimensions in other properties using well-understood materials and methods. Strength testing may be easier to conduct on individual tubes before they are combined or, in other examples, after combination, but taking advantage of their differing lengths.

One merely illustrative example of forming a tube assembly (e.g., any of those described herein) is shown as process (800) in FIG. 15, beginning at START point (810). At conditional block (812), each individual tube is tested for durability and bursting strength. If the test fails, the part is rejected and/or remediated (814). On the other hand, if the test is successful, a plurality of tubes is combined (816) and then the combination is used in a patient procedure (818). Process (800) ends at END point (820). Thus, process (800) provides testing of tubes before they are combined in a tube assembly.

In some other processes, tubes are combined before they are tested. An example of such a process (900) is illustrated in FIG. 15, beginning at START point (910). In process (900), a plurality of single lumens is combined (912), then the individual lumens are tested (conditional block (914)). If the test fails, the item is rejected and/or remediated (916). If, on the other hand, the test succeeds, the device is used in a patient procedure (918), and the process (900) ends at END point (920).

### III. MISCELLANEOUS

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) a plurality of inner tubes, each inner tube having respective lumen extending along a respective axis;
(b) an outer tube extending around at least one of the inner tubes; and
(c) an inflatable balloon coupled with at least one of the lumens of the inner tubes, wherein the lumen associated with the inflatable balloon is configured to communicate fluid to the inflatable balloon;
wherein the axes of the lumens of the inner tubes are substantially parallel to each other and laterally offset from each other along the lengths of the inner tubes;
wherein the combination of the inner tubes, the outer tube, and the inflatable balloon is sized for insertion in an anatomical passageway of a patient.

2. The apparatus of claim 1, wherein the combination of the inner tubes, the outer tube, and the inflatable balloon is sized for insertion in an airway of a patient

3. The apparatus of claim 1, wherein one of the inner tubes has a length greater than the length of at least one of the other inner tubes.

4. The apparatus of claim 1, wherein the outer tube is coaxial with one of the inner tubes.

5. The apparatus of claim 4, wherein another one of the inner tubes is located exterior to the outer tube.

6. The apparatus of claim 1, wherein the outer tube defines a longitudinal axis, wherein the inner tubes are formed in an arrangement centered about the longitudinal axis of the outer tube.

7. The apparatus of claim 1, wherein at least one of the inner tubes comprises a tapered tube having an outer diameter that tapers along the length of the tapered tube.

8. The apparatus of claim 7, wherein the lumen of the tapered tube has a constant diameter along the length of the tapered tube.

9. The apparatus of claim 8, wherein the tapered tube has a wall thickness that varies along the length of the tapered tube.

10. The apparatus of claim 1, wherein the lumen of the tapered tube has a varying diameter along the length of the tapered tube.

11. The apparatus of claim 10, wherein the tapered tube has a substantially constant wall thickness along the length of the tapered tube.

12. The apparatus of claim 1, wherein the outer tube comprises a heat shrink wrap.

13. The apparatus of claim 1, wherein the outer tube defines an interior gap between the interior of the outer tube and the exterior of the inner tubes.

14. The apparatus of claim 13, wherein the interior gap is configured to communicate with a suction source.

15. The apparatus of claim 1, wherein the outer tube includes depth indicia.

16. The apparatus of claim 1, wherein at least one of the inner tubes has a wall with a hardness that varies along the length of the inner tube.

17. The apparatus of claim 1, wherein at least one of the lumens of the inner tubes is sized to receive a stylet or guide wire.

18. An apparatus comprising:
(a) a plurality of inner tubes, each inner tube having respective lumen extending along a respective axis;
(b) an outer layer of shrink wrap extending around the plurality of inner tubes; and
(c) an inflatable balloon coupled with at least one of the lumens of the inner tubes, wherein the lumen associated with the inflatable balloon is configured to communicate fluid to the inflatable balloon;
wherein the axes of the lumens of the inner tubes are substantially parallel to each other;
wherein the combination of the inner tubes, the outer layer of shrink wrap, and the inflatable balloon is sized for insertion in an anatomical passageway of a patient.

19. An apparatus comprising:
(a) a first tube defining a first lumen extending along a first axis;
(b) a second tube defining a second lumen extending along a second axis;
(c) a third tube defining a third lumen extending along a third axis;
(d) an inflatable balloon in fluid communication with the first lumen; and
(e) a guide member slidably disposed in the second lumen;
wherein the first, second, and third axes are parallel with each other;
wherein the first, second, and third axes are laterally offset from each other;
wherein the first, second, and third tubes are secured to each other;
wherein the combination of the first, second, and third tubes are sized for insertion in an anatomical passageway of a patient.

20. The apparatus of claim 19, further comprising an outer member disposed about the first, second, and third tubes, such that the first, second, and third tubes are at least partially contained by the outer member.
